(19) United States
(12) Patent Application Publication
(10) Pub. No.: US 2017/0231780 A1
(43) Pub. Date: Aug. 17, 2017

(54) EXPANDABLE FUSION CAGE

(71) Applicant: Paul S. D'URSO, Richmond, Victoria (AT)

(72) Inventor: Paul S. D'URSO, Richmond, Victoria (AT)

(21) Appl. No.: 15/501,401

(22) PCT Filed: May 20, 2015
(86) PCT No.: PCT/AU2015/050257

§ 371 Date: Feb. 2, 2017

(30) Foreign Application Priority Data
Aug. 5, 2014 (AU) 2014100879

(51) Int. Cl. A61F 2/44; A61F 2/30; A61F 2/46

(52) U.S. Cl. CPC A61F 2/4455; A61F 2/4611; A61F 2/30771; A61F 2002/30131; A61F 2002/30373; A61F 2002/30471; A61F 2002/3052; A61F 2002/30538; A61F 2002/30545; A61F 2002/30904

(57) ABSTRACT

There is provided an intervertebral fusion cage which is expandable in multiple dimensions. The fusion cage finds use in surgery, particularly minimally invasive spinal surgery. There is also provided a method of using the fusion cage in spinal surgery.

### FIELD

The present disclosure relates to an expandable intervertebral fusion cage and methods for use in spinal fusion. The fusion cage is compatible with minimally invasive surgical techniques.

### BACKGROUND

Lumbar fusion surgery has been augmented by the use of intervertebral fusion cages for many years. Typically, such fusion cages are made out of biocompatible materials such as polyether ether ketone or titanium. Fusion cages provide stress shielding to bone graft and provide biomechanical support at the adjacent vertebral bodies whilst bony consolidation of the graft occurs to join the two adjacent vertebral bodies together by way of bony fusion or ossification. One of the limitations of a fusion cage is that the correct size needs to be ascertained prior to its placement. As the two vertebral bodies can move independently of each other, it can be difficult to determine the correct size of the cage.

In minimally invasive lumbar fusion surgery it is difficult to insert a large enough fusion cage. The inability to insert a properly sized fusion cage may prevent satisfactory fusion of the cage to the vertebral bodies and may also increase the risk of post-operative migration of the cage. Both situations may result in reduced efficacy of the surgical treatment.

Prior art interbody fusion cages have approached this problem by providing for expansion of the implant after it is placed into the disc space. A variety of designs of expandable cage have been described, but they are limited to expansion in one plane.

Therefore, it would be desirable to provide a fusion cage that could address the above limitations.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

### SUMMARY

In one aspect there is provided an expandable intervertebral fusion cage said fusion cage being expandable in two or more dimensions. The fusion cage may be expandable in three dimensions. The fusion cage may be simultaneously expandable in two or more dimensions.

The fusion cage may incorporate a larger amount of bone graft in an expanded state compared to a non-expanded state. The fusion cage may have a higher surface area of contact of both bone graft and fusion cage with vertebral bodies in an expanded state compared to a non-expanded state.

The internal volume of the fusion cage in a non-expanded state may be between 0.3 cm³ and 10 cm³, or between 0.5 cm³ and 5 cm³, or between 0.5 cm³ and 3 cm³.

The internal volume of the fusion cage in an expanded state may be between 1 cm³ and 15 cm³ or between 1.5 cm³ and 10 cm³ or between 2 cm³ and 8 cm³.

The fusion cage may expand in volume between about 100% and about 600% based on the volume in the expanded state relative to the volume in the non-expanded state. The fusion cage may expand in volume between about 200% and about 500% based on the volume in the expanded state relative to the volume in the non-expanded state.

The expansion in volume from the non-expanded state to the expanded state may be greater than 100%, or greater than 200%, or greater than 300%, or greater than 400%, or greater than 500%.

The fusion cage may have a volume in the non-expanded state between 0.5 cm³ and 2.5 cm³ and a volume in the expanded state between 1.5 cm³ and 8 cm³.

The fusion cage may have a bone graft volume in the expanded state between about 2 cm³ and about 8 cm³ or between about 4 cm³ and about 7 cm³.

In one embodiment the expandable fusion cage comprises:

a U-shaped upper portion having open proximal ends and a closed distal end, and a longitudinal axis extending therebetween;

a U-shaped lower portion having open proximal ends and a closed distal end, and a longitudinal axis extending therebetween;

a hinge portion separately coupled to the distal ends of the upper and lower portions, the hinge portion having two hinge axes disposed transverse to the longitudinal axes of the upper and lower portions;

an expansion element disposed between the upper and lower portions, and disposed between legs defined by the U-shapes of each of the upper and lower portions, the expansion element adapted for simultaneous slideable movement between the upper and lower portions and between the legs of each of the upper and lower portions.

The fusion cage has a central expansion element, which may move along the axial length of the cage to allow the expansion of the four legs, defined by the upper and lower portions, in more than one plane. Once the appropriate expansion has been determined, the central expansion element may be locked into position.

Each of the upper and lower portions may be, at least partially, curved, so that, in an unexpanded state, the distance between the respective proximal ends of the upper and lower portions is less than the distance between respective central points located longitudinally on the upper and lower portions.

The width of each of the legs of the upper and lower portions may increase from any point along their longitudinal axes to their proximal ends.

The fusion cage may further comprise opposing surfaces defined by a lower surface of the upper portion and an upper surface of the lower portion wherein the opposing surfaces are adapted to engage the expansion element.

The opposing surfaces may comprise one or more slots adapted to engage the expansion element.

The fusion cage may further comprise opposing surfaces defined by the inner surfaces of the upper portion and lower portion, wherein the opposing surfaces are adapted to engage the expansion element.

The opposing surfaces may comprise one or more slots adapted to engage the expansion element.

The hinge portion may be equipped with connection means for connecting to a tool to facilitate placement of the fusion cage in an intervertebral space.

The fusion cage may expand symmetrically.

On moving the expansion element towards the proximal ends of the upper and lower portions, each of the legs of the upper and lower portions may expand outwardly so that the opening defined by the proximal ends of the upper and lower portions may be enlarged.

In another embodiment the expandable fusion cage comprises:

a lower portion having a proximal end and a distal end, and a longitudinal axis extending therebetween;

a U-shaped upper portion having an open proximal end and a closed distal end, and a longitudinal axis extending therebetween;

a hinge portion separately coupled to the proximal end of the lower portion and to an expansion element, the hinge portion having two hinge axes disposed transverse to the longitudinal axes of the upper and lower portions;

wherein the expansion element is disposed between legs defined by the U-shaped upper portion, the expansion element adapted for slideable movement between the legs; and

wherein the distal end of the upper portion is hingedly attached to the distal end of the lower portion.

The width of each of the legs of the upper portion may increase from any point along their longitudinal axes to their proximal ends.

The fusion cage may further comprise opposing surfaces defined by the inner surfaces of the upper portion, wherein the opposing surfaces are adapted to engage the expansion element.

The opposing surfaces may comprise one or more slots adapted to engage the expansion element.

The fusion cage has a hinged expansion element, which may slide along the upper portion of the fusion cage, and as it does so, expands the cage in more than one dimension. Such expansion may be asymmetric, the greater degree of expansion being produced in the upper portion of the cage along which the expansion element moves.

The hereinbefore disclosed fusion cages may allow for multiplanar expansion as well as allowing bone graft to be incorporated into the cage after the cage has been expanded. The fusion cages may facilitate a free surface area of contact between bone graft and adjacent vertebral bodies in the expanded state. The cages may provide structural support to the adjacent vertebral bodies to maintain separation at a desired distance.

It will be appreciated that a variety of angulations may be achieved through the expansion of the cages. A single expandable cage according to the present disclosure may be able to replace several non-expandable cages in achieving satisfactory positioning of the cage and also positioning of bone graft material in the intervertebral space. It may be appreciated by virtue of the design of the cages that a variety of anatomical variations may be conformed to by virtue of asymmetric or symmetric multiple dimensional expansion.

As it is difficult to predict in what way intervertebral expansion will occur prior to surgery, the ability to alter the expansion of a cage at the time of surgery provides an advantage over a traditional non-expandable cage. The ability to expand the cage in multiple dimensions offers significant advantage over currently available expandable cages. The ability to incorporate bone graft into the cage once in an expanded position also is of benefit compared to current expandable cages, which often will not allow bone graft to be placed once they are held in an expanded position. If the cage is expanded without increased amount of bone graft being placed, it is less likely that a successful fusion will be obtained.

The expansion cages may be made of metal or a biocompatible polymer. Exemplary metals include titanium. Exemplary polymers include polyether ether ketone.

In another aspect there is provided a spinal implant system, comprising:
a) an expandable fusion cage as hereinbefore described; and
b) an implantation tool for positioning the fusion cage at a desired location and moving the expansion element so as to expand the cage in two or more dimensions.

In another aspect there is provided a spinal implant system, comprising:
a) an expandable fusion cage as hereinbefore described; and
b) an implantation tool for positioning the fusion cage at a desired location and moving the expansion element to a desired position between the upper and lower portions.

In another aspect there is provided a method of using an expandable fusion cage, comprising:
a) providing an expandable fusion cage as hereinbefore described;
b) inserting the cage between two adjacent vertebrae at a desired angular orientation; and
c) moving the expansion element so as to expand the cage in two or more dimensions.

In another aspect there is provided a method of using an expandable fusion cage, comprising:
a) providing an expandable fusion cage as hereinbefore described;
b) inserting the cage between two adjacent vertebrae at a desired angular orientation; and
c) moving the expansion element to a desired position between the upper and lower portions.

Throughout this specification, use of the terms “comprises” or “comprising” or grammatical variations thereon shall be taken to specify the presence of stated features, integers, steps or components but does not preclude the presence or addition of one or more other features, integers, steps, components or groups thereof not specifically mentioned.

### BRIEF DESCRIPTION OF THE DRAWINGS

FIG. 1 illustrates several views of a fusion cage according to one embodiment of the present disclosure.

FIG. 2 illustrates several views of a fusion cage according to another embodiment of the present disclosure.

FIG. 3 illustrates a tool for facilitating placement of a fusion cage.

### DESCRIPTION OF EXEMPLARY EMBODIMENTS

Before the present devices and/or methods are disclosed and described, it is to be understood that unless otherwise indicated this invention is not limited to specific devices, components, designs, methods, or the like, as such may vary, unless otherwise specified. It is also to be understood that the terminology used herein is for the purpose of describing particular embodiments only and is not intended to be limiting.

It must also be noted that, as used in the specification and the appended claims, the singular forms ‘a’, ‘an’ and ‘the’ include plural referents unless otherwise specified. Thus, for example, reference to ‘a guide wire’ may include more than one guide wires, and the like.

Disclosed herein are advantageous devices and methods for performing spinal surgery, particularly spinal fusion surgery.

Spinal interbody fusion is a process of achieving the union of two vertebral bodies by a bone graft. Typically the development of an interbody fusion is facilitated by a surgical procedure to facilitate a therapeutic outcome in patients that might have a variety of spinal pathological conditions. Such conditions include instability of a particular spinal motion segment, degenerative intervertebral disc condition or other conditions causing derangement of the intervertebral motion segment that result in neural impairment or physical incapacity.

Commonly, surgical interbody fusions are achieved by a posterior or dorsal approach to the spine. Such an exposure usually necessitates removal of posterior elements of the spine to access the intervertebral disc of the selected motion segment. The present disclosure utilises a posterior surgical approach, which allows access to the intervertebral space and intervertebral disc to facilitate the creation of an interbody spinal fusion.

Once the intervertebral or interbody space has been prepared, an illuminated balloon device can be inserted by way of a surgical retractor. Such a balloon may then be inflated by way of insufflation of fluid to assess the volume of the interbody space in relation to the pressure required for such insufflation to occur. The volume of insufflation may be directly correlated to the volume of the interbody space as may the degree of pressure developed to provide such volumetric expansion by way of a pressure sensor or a strain gauge sensor or manometer. Such information may then be used to assist with the selection and delivery of an interbody or intervertebral fusion cage to maintain the distraction or compression of adjacent vertebral bodies to facilitate the process of the interbody fusion. The fusion cage may be inserted through a surgical retractor into the intervertebral space.

The cage may be delivered by way of an access retractor or by way of direct placement and visualisation of the interbody space without the access retractor. It is envisaged that the access retractor may be used either before or after placement of the intervertebral fusion cage to facilitate the placement of bone graft or bone graft substitute or other such types of biological scaffolding or growth related stimulation factors into the interbody space. It is envisaged that the access retractor may carry with it an internal cannulated device to assist with the directional delivery of interbody graft material. Such a cannulated device could come with a predetermined angular delivery aperture within the intervertebral disc or come with a delivery aperture, which would be movable or steerable by the surgeon. In a similar way the access retractor may be used to place an intervertebral fusion cage, which may have an expandable characteristic into the intervertebral space. Such a cage would be steered into a desired position using medical imaging in real time to determine the location of such a cage in the intervertebral space. It is envisaged that once the cage was placed in a satisfactory position a secondary mechanism may be initiated to allow expansion of the cage in a predetermined way to allow the incorporation of an increased volume of bone graft or bone graft substitute as well as to deliver a biomechanical force to the adjacent vertebral bodies in a predetermined way.

Once the access retractor is placed into the intervertebral space an automated process may be initiated in such a way that the intervertebral discectomy and preparation of the end plates, removal of debris, inspection of the adequacy of preparation and subsequent delivery of bone graft or bone graft substitute in the intervertebral fusion cage could occur in an automated fashion which would facilitate the procedure being performed by a robotic apparatus.

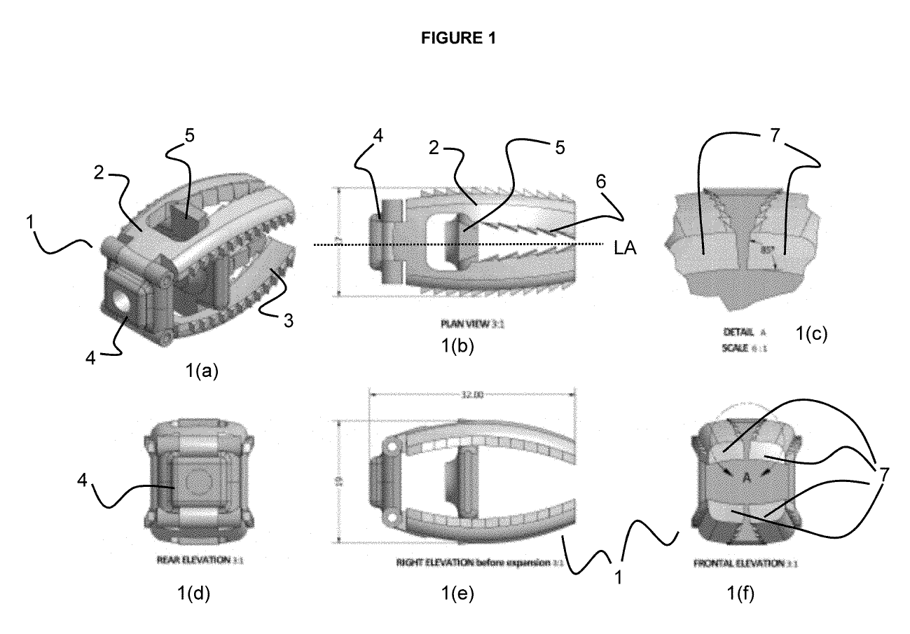

One embodiment of a fusion cage according to the present disclosure is shown in FIG. 1. FIG. 1(a) illustrates cage (1) having upper portion (2) and lower portion (3), each of an approximate U-shape and each coupled to hinge portion (4). Expansion element (5) is located between the upper and lower portions. In operation, the expansion element (5) moves from the distal end to the proximal end and thus expands each leg of the upper and lower portions.

FIG. 1(b) shows a plan view of the expansion cage having upper portion (2), hinge section (4) and expansion element (5). The upper portion has slots (6) which engage with the expansion element. The longitudinal axis is represented by the dotted line ‘LA’.

FIG. 1(c) shows a detailed view of the proximal ends (7) of the legs of the upper portion.

FIG. 1(d) shows a rear elevation of the cage and hinge portion (4).

FIG. 1(e) is a side elevation of expansion cage (1) in unexpanded form.

FIG. 1(f) shows a front elevation of expansion cage (1) illustrating each leg of the upper and lower portions and their proximal ends (7).

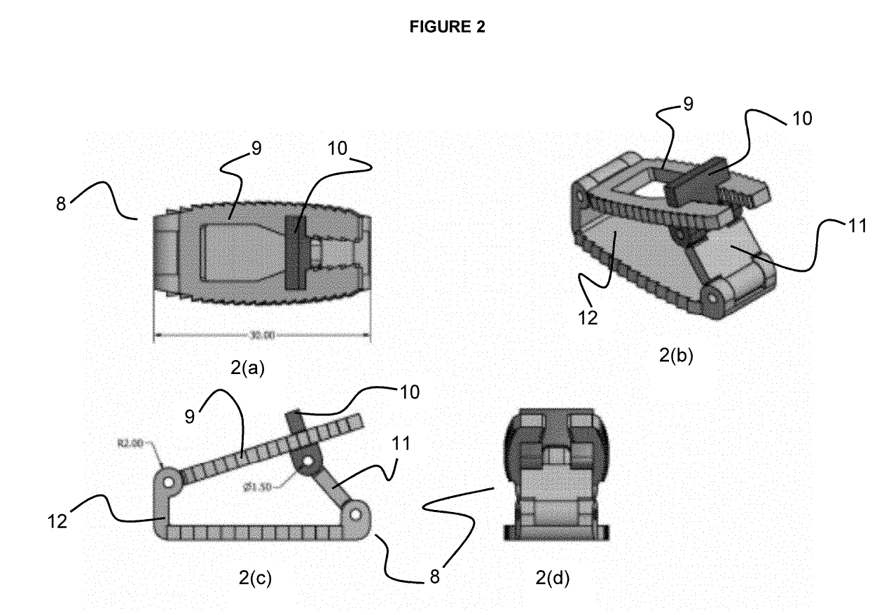

Another embodiment of a fusion cage according to the present disclosure is shown in FIG. 2. FIG. 2(a) shows a plan view of fusion cage (8) having upper portion (9) and expansion element (10). FIG. 2(b) additionally shows hinge portion (11) and lower portion (12). FIG. 2(c) is a side elevation of fusion cage (8) and FIG. 2(d) a front elevation.

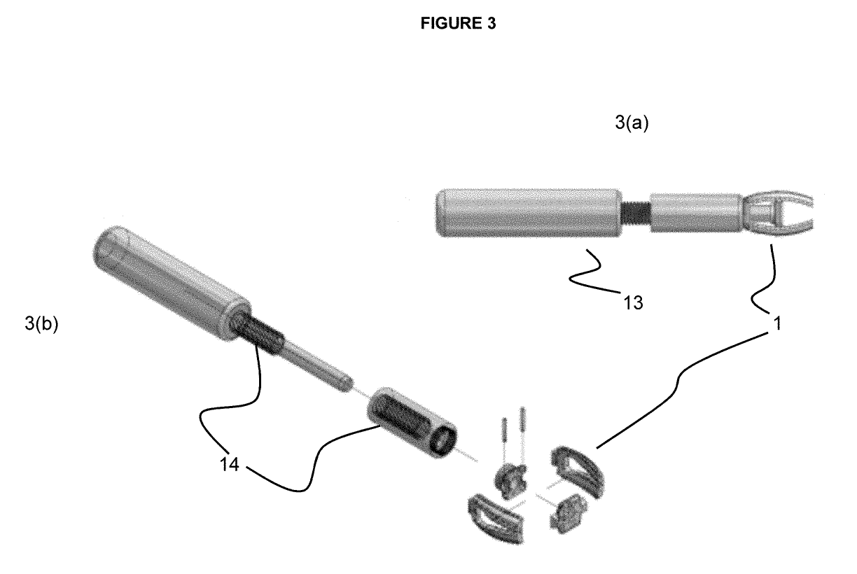

A tool for placement of a fusion cage according to the present disclosure is shown in FIG. 3. Tool (13) is illustrated in FIG. 3(a) with coupled fusion cage (1). FIG. 3(b) is an exploded view of tool (13) and cage (1). The tool has an internal spring assembly (14) for uncoupling the cage for placement in an intervertebral position.

It is to be understood that while the present disclosure has been described in conjunction with the specific embodiments thereof, the foregoing description is intended to illustrate and not limit the scope of the disclosure. Other aspects, advantages and modifications will be apparent to those skilled in the art to which the disclosure pertains. Therefore, the following examples are put forth so as to provide those skilled in the art with a complete disclosure and description of how to make and use the disclosed compositions, and are not intended to limit the scope of the disclosure.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited.

All documents cited are herein fully incorporated by reference for all jurisdictions in which such incorporation is permitted and to the extent such disclosure is consistent with the description of the present disclosure.

1. An expandable intervertebral fusion cage, said fusion cage being expandable in two or more dimensions.

2. (canceled)

3. (canceled)

4. A fusion cage according to claim 1, wherein the internal volume of the fusion cage in a non-expanded state is between 0.3 cm³ and 10 cm³.

5. A fusion cage according claim 1, wherein the internal volume of the fusion cage in an expanded state is between 1 cm³ and 15 cm³.

6. (canceled)

7. A fusion cage according to claim 1, wherein the expansion in volume from a non-expanded state to an expanded state is greater than 200%.

8. A fusion cage according to claim 1 comprising:
a U-shaped upper portion having open proximal ends and a closed distal end, and a longitudinal axis extending therebetween;
a U-shaped lower portion having open proximal ends and a closed distal end, and
a longitudinal axis extending therebetween;
a hinge portion separately coupled to the distal ends of the upper and lower portions, the hinge portion having two hinge axes disposed transverse to the longitudinal axes of the upper and lower portions; and
an expansion element disposed between the upper and lower portions, and disposed between legs defined by the U-shapes of each of the upper and lower portions, the expansion element adapted for simultaneous slideable movement between the upper and lower portions and between the legs of each of the upper and lower portions.

9. A fusion cage, according to claim 8, wherein each of the upper and lower portions are, at least partially, curved, so that, in an unexpanded state, the distance between the respective proximal ends of the upper and lower portions is less than the distance between respective central points located longitudinally on the upper and lower portions.

10. A fusion cage according to claim 8, wherein the width of each of the legs of the upper and lower portions increases from any point along their longitudinal axes to their proximal ends.

11. A fusion cage according to claim 8 further comprising opposing surfaces defined by a lower surface of the upper portion and an upper surface of the lower portion wherein the opposing surfaces are adapted to engage the expansion element.

12. A fusion cage according to claim 11, wherein the opposing surfaces comprise one or more slots adapted to engage the expansion element.

13. A fusion cage according to claim 11 further comprising opposing surfaces defined by the inner surfaces of the upper portion and lower portion wherein the opposing surfaces are adapted to engage the expansion element.

14. A fusion cage according to claim 13, wherein the opposing surfaces comprise one or more slots adapted to engage the expansion element.

15. A fusion cage according to claim 8, wherein expansion of the cage is symmetric.

16. A fusion cage according to claim 8, wherein on moving the expansion element towards the proximal ends of the upper and lower portions, each of the legs of the upper and lower portions expand outwardly so that the opening defined by the proximal ends of the upper and lower portions is enlarged.

17. A fusion cage according to claim 1 comprising:
a lower portion having a proximal end and a distal end, and a longitudinal axis extending therebetween;
a U-shaped upper portion having an open proximal end and a closed distal end, and a longitudinal axis extending therebetween; and
a hinge portion separately coupled to the proximal end of the lower portion and to an expansion element, the hinge portion having two hinge axes disposed transverse to the longitudinal axes of the upper and lower portions;
wherein the expansion element is disposed between legs defined by the U-shaped upper portion, the expansion element adapted for slideable movement between the legs; and
wherein the distal end of the upper portion is hingedly attached to the distal end of the lower portion.

18. A fusion cage according to claim 17, wherein the width of each of the legs of the upper portion increases from any point along their longitudinal axes to their proximal ends.

19. A fusion cage according to claim 17 further comprising opposing surfaces defined by the inner surfaces of the upper portion wherein the opposing surfaces are adapted to engage the expansion element.

20. A fusion cage according to claim 19, wherein the opposing surfaces comprise one or more slots adapted to engage the expansion element.

21. A fusion cage according to claim 17, wherein on sliding the hinged expansion element along the upper portion of the fusion cage, the cage expands the in more than one dimension.

22. A fusion cage according to claim 21, wherein the expansion is asymmetric, the greater degree of expansion being produced in the upper portion of the cage along which the expansion element moves.

23. (canceled)

24. (canceled)

25. (canceled)

26. A spinal implant system, comprising:
a) an expandable intervertebral fusion cage according to claim 1; and
b) an implantation tool for positioning the fusion cage at a desired location and moving the expansion element so that the cage expands in two or more dimensions.

27. A spinal implant system, comprising:
a) an expandable intervertebral fusion cage according to claim 1; and
b) an implantation tool for positioning the fusion cage at a desired location and moving the expansion element to a desired position between the upper and lower portions.

28. (canceled)

29. (canceled)
